(19) United States
(12) Patent Application Publication
(10) Pub. No.: US 2023/0310023 A1
(43) Pub. Date: Oct. 5, 2023

(54) COLD SNARE

(71) Applicant: MICRO-TECH (NANJING) CO., LTD., Nanjing (CN)

(72) Inventors: Jianyu WEI, Nanjing (CN); Zhenghua SHEN, Nanjing (CN); Feng ZHANG, Nanjing (CN); Changqing Ll, Nanjing (CN); Derong LENG, Nanjing (CN); Scott HAACK, Nanjing (CN); Zhi TANG, Nanjing (CN); Zhengping HOU, Nanjing (CN); Lei WU, Nanjing (CN); Hongyan JIN, Nanjing (CN)

(21) Appl. No.: 18/332,537

(22) Filed: Jun. 9, 2023

Related U.S. Application Data
(63) Continuation of application No. PCT/CN2021/137561, filed on Dec. 13, 2021.

(30) Foreign Application Priority Data
Dec. 15, 2020 (CN) 202011484951.2

(51) Int. Cl. A61B 17/3205

(52) U.S. Cl. CPC A61B 17/32056; A61B 17/320016

(57) ABSTRACT

A cold snare includes a spring tube, a drive wire, and a loop. The cable passes through the spring tube, and can perform reciprocating motion in a direction of a longitudinal axis of the spring tube. The loop is fixedly connected to a distal end of the drive wire. Therefore, when the drive wire performs reciprocating motion in the direction of the longitudinal axis of the spring tube, the loop synchronously performs reciprocating motion in the direction of the longitudinal axis of the spring tube along with the drive wire to achieve the purpose of controlling the loop to enter and exit the spring tube by the drive wire. By using a spring tube as an outer tube, the strength of the spring tube is higher than that of a plastic outer tube of an existing cold snare, which is helpful for the loop to cut when grabbing large polyps.

### CROSS-REFERENCE TO RELATED APPLICATION

This application is a continuation of International Application No. PCT/CN2021/137561, filed on Dec. 13, 2021, which claims priority to Chinese Patent Application No. 202011484951.2, filed on Dec. 15, 2020. The aforementioned applications are hereby incorporated by reference in their entireties.

### TECHNICAL FIELD

The present disclosure relates to the field of medical devices, and in particular, to a cold snare.

### BACKGROUND

Endoscopic mucosal resection (EMR) is a technique of injecting a drug into the submucosa of lesions (such as sessile polyps, flat or shallow sunken polyps, leiomyoma, early carcinoma of esophagus, stomach, colon, etc.) to form a liquid pad and cutting off a large piece of mucosal tissue.

Snare, which is a commonly used surgical device for EMR, includes cold snare and hot snare. The hot snare is used to perform polyp cutting with electricity during use by setting up a high-frequency power supply. However, a traditional electric snare for polyp removal is often accompanied by perforation, intraoperative and postoperative bleeding, burning the patient and other risk, and meanwhile, the polyp removed by the electric snare is burned, which is difficult to meet adequate histological examination. The cold snare, which is used without electricity, is to cut only through a cutting force of loop itself, which can effectively avoid the risk of burning the tissue and burning the patient.

However, the cutting force of the existing cold snare is weaker than that of the hot snare, and the cutting is easy to produce deformation when grabbing large polyps, leading to the problem of failure to cut.

### SUMMARY

The present disclosure provides a cold snare, including: a spring tube; a drive wire, passing through the spring tube, and reciprocating motion in a direction of a longitudinal axis of the spring tube; and a loop, fixedly connected to a distal end of the drive wire, motion of the drive wire being used for controlling the loop to enter and exit the spring tube.

In an implementation, the cold snare further includes an end member fixedly connected to a distal end of the spring tube, the end member has an inner hole in communication with the spring tube; the loop enters and exits the spring tube through the inner hole.

In an implementation, the end member has a distal end face and an inner circumferential surface corresponding to the inner hole, and a round corner is arranged between the distal end face and the inner circumferential surface.

In an implementation, the end member is made of a metal.

In an implementation, the loop is made of a nickel-titanium alloy.

In an implementation, the loop is in a single wire structure.

In an implementation, a cross section of the wire is round, square or flat.

In an implementation, the loop includes a first ring segment, a necking segment and a second ring segment connected sequentially; the first ring segment has a first intersection point connected with the necking segment, and the first ring segment is in a streamlined shape with smooth transition from a proximal end of the first ring segment to the first intersection point; the second ring segment has a second intersection point connected with the necking segment, and the second ring segment is in a streamlined shape with smooth transition from a proximal end of the second ring segment to the second intersection point.

In an implementation, the loop is water drop-shaped or oval.

In an implementation, the cold snare further includes a riveted tube, the riveted tube has a cylindrical outer circumferential surface, a distal end of the drive wire is fixedly connected in the riveted tube, and a proximal end of the loop is fixedly connected in the riveted tube.

In an implementation, the cold snare further includes an outer skin wrapped on outside of the spring tube.

In an implementation, the outer skin includes a polymer tube.

In an implementation, the distal end of the spring tube is further provided with a scale for measuring a size of a polyp to be cut.

In an implementation, the spring tube includes a flexible metal tube or an elastic metal tube.

In an implementation, the flexible metal tube or elastic metal tube comprises any one of a metal wire wound spring tube and a spirally cut metal tube, such as a spirally cut stainless steel tube, or a combination thereof.

### BRIEF DESCRIPTION OF DRAWINGS

To describe the technical solutions in embodiments of the present disclosure more clearly, the following briefly introduces the accompanying drawings needed for describing the embodiments. It should be understood that the following accompanying drawings illustrate merely some embodiments of the present disclosure, and thus should not be regarded as limiting the scope, and those skilled in the art may still derive other related drawings from these accompanying drawings without creative effort.

FIG. 1 is a schematic diagram of an overall structure of a cold snare provided by some embodiments of the present disclosure.

FIG. 2 is a schematic sectional view of local structure of a cold snare provided by some embodiments of the present disclosure.

FIG. 3 is a schematic diagram of sectional structure of an end member of a cold snare provided by some embodiments of the present disclosure.

FIG. 4 is a schematic structural diagram of a loop of a cold snare provided by some embodiments of the present disclosure.

FIG. 5 is a schematic diagram of a local structure of a loop in a half-open state in a cold snare provided by some embodiments of the present disclosure.

FIG. 6 is a schematic diagram of a local structure of a loop in a retracted state in a cold snare provided by some embodiments of the present disclosure.

FIG. 7 is an enlarged schematic diagram of a local structure at VII in FIG. 1.

FIG. 8 is a schematic structural diagram of a metal wire wound spring tube in some embodiments of the present disclosure.

FIG. 9 is a schematic structural diagram of a spirally cut stainless steel tube in some embodiments of the present disclosure.

FIG. 10 is a schematic structural diagram of a combination of a metal wire wound spring tube and a spirally cut stainless steel tube in some embodiments of the present disclosure.

Reference signs: 100—cold snare; 111—spring tube; 112—outer skin; 113—scale; 1111—metal wire wound spring tube; 1112—spirally cut stainless steel tube; 120—handle; 121—front handle; 122—rear handle; 130—drive wire; 140—loop; 141—connecting part; 142—fetching part; 143—first ring segment; 144—necking segment; 145—second ring segment; 146—first intersection point; 147—second intersection point; 150—end member; 151—inner hole; 152—round corner; 153—distal end face; 160—riveted tube; 170—longitudinal axis.

### DESCRIPTION OF EMBODIMENTS

To make the objects, technical solutions, and advantages of embodiments of the present disclosure clearer, the following clearly and comprehensively describes the technical solutions in embodiments of the present disclosure with reference to the accompanying drawings in the embodiments of the present disclosure. Apparently, the described embodiments are merely some rather than all embodiments of the present disclosure. Usually, components in the embodiments of the present disclosure described and shown in the accompanying drawings herein may be arranged and designed in various configurations.

Therefore, the following detailed description of the embodiments of the present disclosure provided in the accompanying drawings is not intended to limit the scope of the present disclosure for protection, but merely represents the selected embodiments of the present disclosure. All other embodiments obtained by those skilled in the art based on embodiments of the present disclosure without creative effort shall fall within the protection scope of the present disclosure.

It should be noted that: similar labels and letters represent similar items in the below accompanying drawings, therefore, once an item is defined in one accompanying drawing, it does not need to be further defined and explained in the subsequent accompanying drawings.

In the description of the present disclosure, it should be clarified that if the orientation or position relationship indicated by the terms “up”, “down”, “inside” and “outside” is based on the orientation or position relationship shown in the accompanying drawings, or the orientation or position relationship that is commonly placed when the disclosed product is used, it is only for the purpose of describing the present disclosure and simplifying the description, rather than indicating or implying that the device or element referred to must have a specific orientation, or be constructed and operated in a specific direction, and therefore, it cannot be understood as a limitation of the present disclosure.

In addition, if the terms “first” and “second” appear, they are only used to distinguish descriptions and cannot be understood as indicating or implying relative importance.

It should be clarified that features in the embodiments of the present disclosure may be combined with each other without conflict.

Snare is a common medical instrument for endoscopic polyp excision in vivo, and perform polyp cutting by means of a loop 140. In some typical embodiments, the snare roughly includes cold snare 100 and hot snare. The hot snare is used to perform polyp cutting with electricity during use by setting up a high-frequency power supply. However, the traditional the electric snare for polyp removal is often accompanied by perforation, intraoperative and postoperative bleeding, burning the patient and other risk, and meanwhile, the polyp removed by the electric snare is burned, which is difficult to meet adequate histological examination. The cold snare 100, which is used without electricity, is to cut only through a cutting force of the loop 140 itself, which can effectively avoid the risk of burning the tissue and burning the patient. However, the cutting force of the existing cold snare 100 is weaker than that of the hot snare, and the cutting is easy to produce deformation when grabbing large polyps, leading to the problem of failure to cut.

The present embodiment provides a cold snare 100, which can effectively alleviate the technical problem that the cold snare 100 is unable to perform cutting of large polyp, the cold snare 100 provided by the present embodiment will be introduced below.

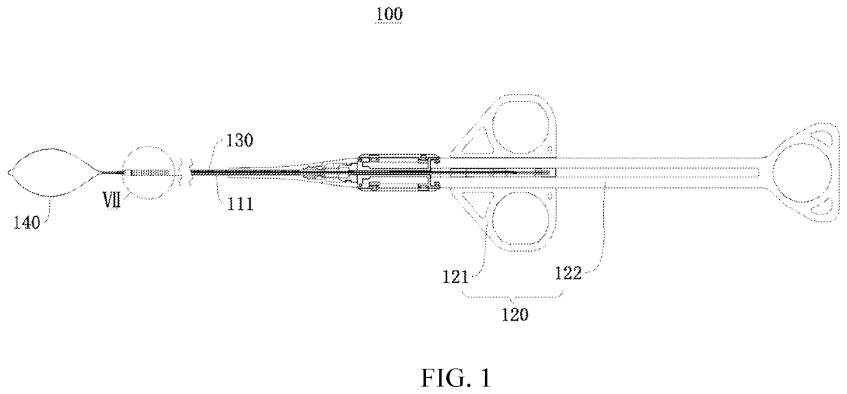

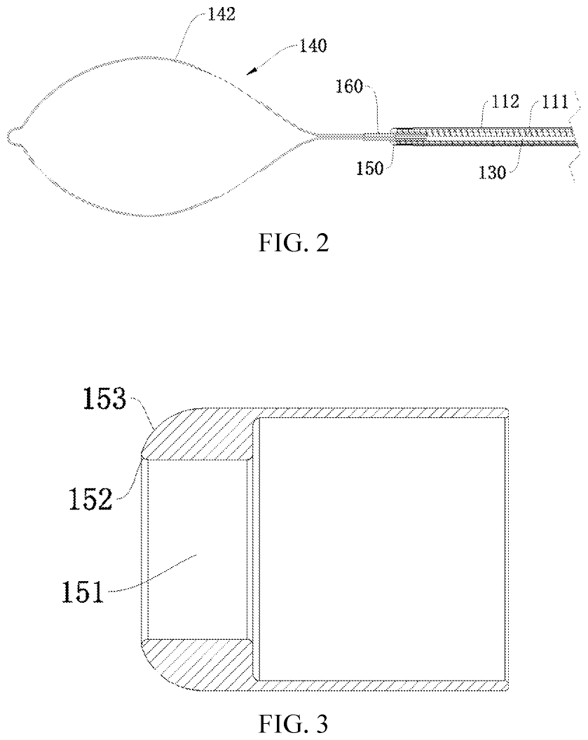

FIG. 1 is a schematic diagram of an overall structure of a cold snare 100 provided by some embodiments of the present disclosure, and FIG. 2 is a sectional diagram of a local structure of the cold snare 100 provided by some embodiments of the present disclosure. Referring to FIGS. 1 and 2, the present embodiment provides a cold snare 100 including a spring tube 111, a drive wire 130, and a loop 140. The drive wire 130 passes through the spring tube 111, and can perform reciprocating motion in a direction of a longitudinal axis 170 of the spring tube 111. The loop 140 is fixedly connected to a distal end of the drive wire 130, so that when the drive wire 130 performs reciprocating motion in the direction of a longitudinal axis 170 of the spring tube 111, the loop 140 performs reciprocating motion synchronously with the drive wire 130 in the direction of the longitudinal axis 170 of the spring tube, thereby achieving the purpose of controlling the loop 140 to enter and exit the spring tube 111 at the edge sealed distal end of the spring tube 111 by the drive wire 130. When the loop 140 extends out of the spring tube 111, the loop 140 opens to form a ring structure enclosing the polyp, and then the loop 140 is controlled to move into the spring tube 111 through the drive wire 130, so that the size of the ring structure formed by the loop 140 is reduced, and then the polyp is clamped inside thereof, and finally realize the cutting of the polyp. Since the cold snare 100 uses the spring tube 111 as the outer tube, the strength of the spring tube 111 is higher than that of the plastic outer tube of the existing cold snare 100, so it is helpful for the loop 140 to perform cutting when grabbing large polyps. At the same time, since the spring tube 111 is made of metal, the friction force generated by the rotation of the loop 140 is smaller than that of an existing plastic outer tube contacting the loop 140, so it is helpful to realize the synchronous and accurate rotation of the loop 140, which is convenient for a doctor to accurately enclose the polyp during operation and improve the use effect, thereby improving the technical problem that the existing cold snare 100 cannot cut a large polyp.

It should be clarified that in the description of the present embodiment, “proximal end” of a part refers to an end of the part relatively close to an outside of the human body when the cold snare 100 is in use, and correspondingly, “distal end” of a part refers to an end of the part relatively close to the inside of the human body when the cold snare 100 is in use. For example, the distal end of the spring tube 111 is an end of the spring tube 111 that lies inside the human body when it is in use, and the proximal end of the spring tube 111 is an end of the spring tube 111 that lies outside the human body when it is in use.

In some embodiments, the outer skin 112 includes, but is not limited to, a polymer tube. The embodiment of the present disclosure does not specifically limit the polymer tube, and the polymer tube may be, for example, a polymer polyethylene tube or an ultra-high molecular polyethylene steel skeleton composite tube.

In some embodiments, the spring tube 111 includes a flexible metal tube or an elastic metal tube. In some embodiments, the spring tube 111 includes a flexible metal tube or an elastic metal tube, and an out layer of the spring tube 111 is coated with a polymer tube.

In some embodiments, the present disclosure does not specifically limit the flexible metal tube or the elastic metal tube, and spring tube 111 may include any one type of flexible metal tubes or elastic metal tubes known in the art, or any combination of any two or more types of flexible metal tubes or elastic metal tubes known in the art. In some embodiments, a combining mode of any combination of any two or more types of flexible metal tubes or elastic metal tubes includes, for example, welding and bonding.

In some embodiments, the flexible metal tube or the elastic metal tube includes, but is not limited to, any one of, for example, metal wire wound spring tube 1111 and spirally cut metal tube such as spirally cut stainless steel tube 1112, or a combination of both.

In some embodiments, a spirally cut metal tube, such as a spirally cut stainless steel tube 1112, is a hypotube.

In some embodiments, the spring tube 1111 consists of a metal wire wound spring tube 1111.

In some embodiments, the spring tube 111 consists of a spirally cut metal tube such as a spirally cut stainless steel tube 1112.

In some typical embodiments, the spring tube 111 consists of a metal wire wound spring tube 1111 and a spirally cut metal tube such as a spirally cut stainless steel tube 1112, wherein either end or both ends of the metal wire wound spring tube 1111 are connected to a spirally cut metal tube such as a spirally cut stainless steel tube 1112.

In some typical embodiments, the spring tube 111 consists of a metal wire wound spring tube 1111 and a spirally cut metal tube such as a spirally cut stainless steel tube 1112, wherein both ends of a spirally cut metal tube such as a spirally cut stainless steel tube 1112 are connected to the metal wire wound spring tube 1111.

The cold snare 100 provided by the present embodiment is further explained below.

Referring to FIG. 1, in the present embodiment, the cold snare 100 further includes a handle 120, and the manipulation of the loop 140 is achieved by operating the handle 120 during use. In some typical embodiments, the handle 120 includes a front handle 121 and a rear handle 122. The front handle 121 is sleeved outside the rear handle 122, and the front handle 121 slides relative to the rear handle 122. The proximal end of the spring tube 111 is connected to the distal end of the rear handle 122, and the proximal end of the drive wire 130 is fixedly connected to the front handle 121. By operating the front handle 121 moves relative to the rear handle 122, the drive wire 130 is driven to move relative to the spring tube 111, thereby realizing the manipulation of the loop 140.

In an implementation, the material of the spring tube 111 is stainless steel, and it is understandable that in other embodiments, the spring tube 111 can also be made of other metal material, as long as it can be ensured that when the loop 140 moves relative to the spring tube 111, the friction between the spring tube 111 and the loop 140 is small enough and the loop 140 has high rotational synchronization.

FIG. 3 is a schematic diagram of sectional structure of an end member 150 of the cold snare 100 provided by the present embodiment. Referring to FIGS. 1-3, in the present embodiment, the cold snare 100 further includes an end member 150 fixedly connected to the distal end of the spring tube 111. The end member 150 is tubular and has an inner hole 151 communicated with the spring tube 111, and the loop 140 enters and exits the spring tube 111 through the inner hole 151. Meanwhile, when the loop 140 enters and exits the spring tube 111, the loop 140 abuts against a wall surface of the inner hole 151, so as to change a radial dimension of the loop 140. It should be clarified that in the description of the present embodiment, “radial dimension of the loop 140” is a distance between the loop 140 and the longitudinal axis 170. In the structure of the end member 150 which provides the edge sealing of the distal end of the spring tube 111 shown in FIG. 3, the proximal end holds the distal end of the spring tube 111 and the distal end is a contraction port, which can increase the cutting force of the cold snare 100 (the smaller the inner diameter of the distal end of the spring tube 111, the stronger the cutting force).

In some typical embodiments, the end member 150 is fixed to the distal end of the spring tube 111 by laser welding, thereby achieving the edge sealing of the distal end of the spring tube 111, which can achieve the effect of not damaging endoscopic forceps channel. And the contraction of the spring tube 111 provided by the distal end of end member 150 can tighten the loop for better tissue cutting. In an implementation, the end member 150 is made of metal. Compared with a plastic material, when the end member 150 is made of metal, there is less friction between the end member 150 and the loop 140 when the end member 150 moves relative to the loop 140, which helps the loop 140 to rotate synchronously, ensuring accurate enclosure of the polyp and improving the use effect. In an implementation, the material of the end member 150 is stainless steel. It is understandable that in other embodiments, the end member 150 can also be made of other metal material, as long as it can be ensured that when the loop 140 moves relative to the end member 150, the friction between the end member 150 and the loop 140 is small enough and the loop 140 has high rotational synchronization.

In an implementation, the end member 150 has a distal end face 153 and an inner circumferential surface corresponding to the inner hole 151, and a round corner 152 is arranged between the distal end face 153 and the inner circumferential surface. In some typical embodiments, the inner circumferential surface of the end member 150 is the circumferential wall of the inner hole 151, and a round corner 152 is arranged between the inner circumferential wall and the distal end face 153 of the end member 150, namely, the inner circumferential wall transitions to the distal end face 153 through the round corner 152, on the basis of which the friction barrier of the loop 140 moving relative to the end member 150 is reduced. In an implementation, the distal end face 153 of the end member 150 is a spherical surface.

Referring again to FIG. 2, in the present embodiment, the cold snare 100 further includes an outer skin 112 covering the outside of the spring tube 111. In other words, the outer tube of the cold snare 100 includes the spring tube 111 and the outer skin 112. By cladding the spring tube 111 with an outer skin 112, the outer surface of the outer tube is smoother and can be easily extended into the human body. In an implementation, the outer skin 112 is made of plastic.

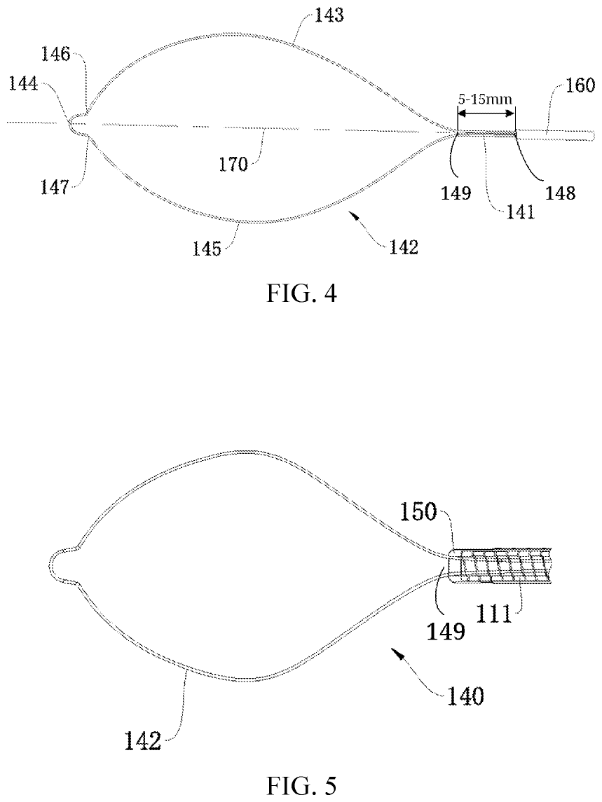

FIG. 4 is a schematic structural diagram of the loop 140 of the cold snare 100 provided by the present embodiment, and FIG. 5 is a schematic diagram of the local structure of the loop 140 in a half-open state in the cold snare 100 provided by the present embodiment. Referring to FIGS. 1-5, in the present embodiment, the loop 140 is made of a nickel-titanium alloy. The nickel-titanium alloy has a memory property, so it can achieve flexible and slow reduction of the radial dimension of the loop 140, while maintaining its original shape during the reduction process, which is helpful to enclose polyps at any time at different diameters.

Therefore, during practical use of the cold snare 100, if it is necessary to radially remove polyps with different diameters, there is no need to replace the snare. For example, if a new polyp is found during further examination after removal of one polyp, and the new polyp has a large size difference from the previous polyp, the removal can be completed without replacing it with a new size of snare. For example, a loop 140 in a fully open state as shown in FIG. 2 is used for cutting a large polyp, and a loop 140 in a semi-open state as shown in FIG. 5 is used for cutting a small polyp, which helps to shorten the operation time and reduce the operation cost.

In an implementation, the loop 140 is in a single wire structure. Most of the existing loops 140 are made of multi-stranded stainless steel wires. Since a nickel-titanium monofilament has a greater strength than the multi-stranded stainless steel wires, it can hold the polyp well. Meanwhile, the nickel-titanium monofilament has a smaller diameter than the multi-stranded stainless steel wires, which makes it sharper when cutting the polyp, and on this basis, it is more helpful to cut large polyps.

In an implementation, the wire has circular cross section, i.e., the cross section of the nickel-titanium monofilament is circular. Understandably, in other embodiments, the cross-sectional shape of the nickel-titanium monofilament can also be set as required, for example, the cross-sectional shape of the nickel-titanium monofilaments can be set as square or flat. In an implementation, the diameter of the nickel-titanium monofilament in the present embodiment is 0.18 mm. Understandably, in other embodiments, the diameter of the nickel-titanium monofilament can also be set as required.

The cold snare 100 provided in the present embodiment greatly improves the sizes of polyps to be cut by the cold snare 100 by setting the structure of the cold snare 100. Compared with the traditional cold snare 100, which can only cut polyps with an external diameter of less than 9 mm, the cold snare 100 with nickel-titanium monofilament provided by the present disclosure can achieve the cutting of polyps with a maximum external diameter of 25 mm.

Continuing to refer to FIGS. 1-5, in the present embodiment, the loop 140 includes a first ring segment 143, a necking segment 144 and a second ring segment 145 connected sequentially. The first ring segment 143 has a first intersection point 146 connected with the necking segment 144, and the second ring segment 145 has a second intersection point 147 connected with the necking segment 144. The first ring segment 143 is in a streamlined shape with smooth transition from a proximal end of the first ring segment 143 to the first intersection point 146, and the second ring segment 145 is in a streamlined shape with smooth transition from a distal end of the second ring segment 145 to the second intersection point 147.

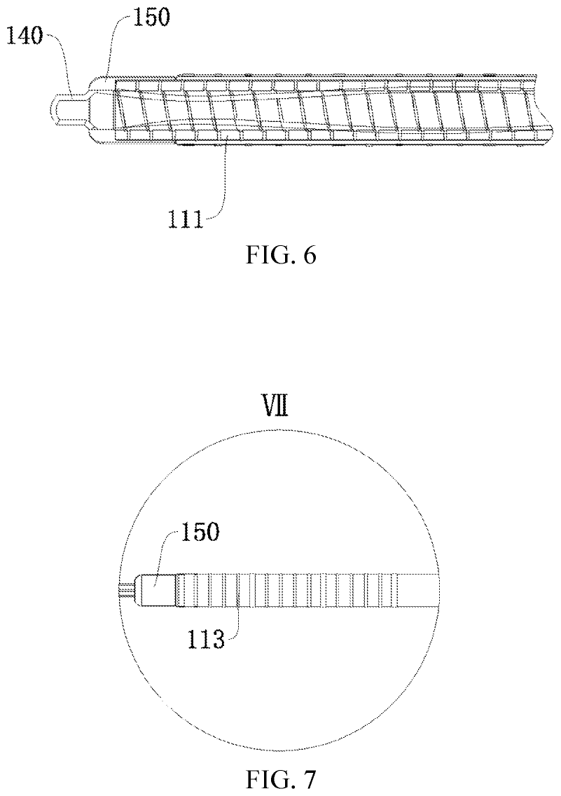

In some typical embodiments, the loop 140 includes a connecting part 141 located at the proximal end and a fetching part 142 located at the distal end, the connecting part 141 is fixedly connected to the distal end of the drive wire 130, and the fetching part 142 is a ring structure that becomes larger in radial dimension to enable enclosing polyps in the process of the loop 140 entering and exiting the end member 150 (as shown in FIGS. 2 and 5), or becomes smaller in radial dimension to be retracted into the spring tube 111 (as shown in FIG. 6). The fetching part 142 includes a first ring segment 143, a necking segment 144 and a second ring segment 145 connected sequentially, and the proximal end of the first ring segment 143 and the proximal end of the second ring segment 145 are connected with the connecting part 141. The first ring segment 143 and the second ring segment 145 are opposite to each other at both sides of the longitudinal axis 170, and the first ring segment 143 and the second ring segment 145 are symmetrically arranged relative to the longitudinal axis 170. By setting the first ring segment 143 and the second ring segment 145 to be streamlined, the loop 140 made of nickel-titanium alloy can be smoothly retracted into the outer tube to prevent the problem of lodging when retracting into the outer tube.

It should be clarified that, in the description of the present embodiment, “streamlined shape” refers to a smooth curve, which does not have a straight line segment or an inflection point, that is, the first derivative of the function corresponding to the curve is continuous, and the centers of the curvature circles on the curve are located on the same side of the curve.

In the present embodiment, the loop 140 is roughly water drop shaped, that is, in the structure shown in FIG. 2, the first ring segment 143 and the second ring segment 145 enclose to form a water drop shape. Due to memory of the nickel-titanium alloy, the diameter of the loop 140 can be reduced flexibly and slowly when pulling the loop 140 to proximally slide relative to the spring tube 111, and the loop 140 always maintains the water drop shape, thereby realizing the purpose of enclosing polyps at any time at different radial dimensions. It is understandable that, in other embodiments, the loop 140 can also be set in an oval shape, as long as it can improve the lodging problem of the loop 140 made of nickel-titanium alloy when retracting into the spring tube 111.

Referring again to FIG. 4, in the present embodiment, the cold snare 100 further includes a riveted tube 160. In some typical embodiments, the riveted tube 160 is arranged in the inner cavity of the spring tube 111. The riveted tube 160 can reciprocate along the longitudinal axis 170 of the spring tube 111. In some typical embodiments, the outer circumferential surface of the riveted tube 160 is a cylindrical surface. The distal end of the drive wire 130 is fixedly connected into the riveted tube 160, and the proximal end of the loop 140 is fixedly connected into the riveted tube 160. In some typical embodiments, the riveted tube 160 is tubular as a whole, and its outer circumference surface is a smooth cylindrical surface. The distal end of the drive wire 130 is fixedly connected with the riveted tube 160, and the distal end of the drive wire 130 is located in the riveted tube 160. The connecting part 141 of the loop 140 is fixedly connected with the riveted tube 160, and the connecting part 141 of the loop 140 is located in the riveted tube 160. The first ring segment 143 and the second ring segment 145 of the loop 140 are merged at the distal end of the riveted tube 160 and are fixed by the riveted tube 160, to form a merger point 148, as shown in FIG. 4, the loop 140 has an opening 149, the opening 149 is located at a place where the first ring segment 143 and the second ring segment 145 begin to enter the end member 150, as shown in FIG. 5, and the opening 149 is a point from which the loop 140 begins to gradually open up to enclose the polyp to be cut. A distance between the merger point 148 and the opening 149 is 5-15 mm, which can ensure that the loop 140 is completely opened in the human body.

FIG. 7 is an enlarged schematic diagram of a local structure at VII in FIG. 1. Referring to FIGS. 1 and 7, in the present embodiment, the distal end of the spring tube 111 is further provided with a scale 113, which is used to measure the size of polyps to be cut, so as to facilitate the cutting operation.

In some embodiments, the outer skin 112 is a polymer tube.

In some embodiments, the spring tube 111 includes a flexible metal tube or an elastic metal tube.

In some embodiments, the flexible metal tube or the elastic metal tube includes but is not limited to either or a combination of metal wire wound spring tube 1111 and a spirally cut stainless steel tube 1112.

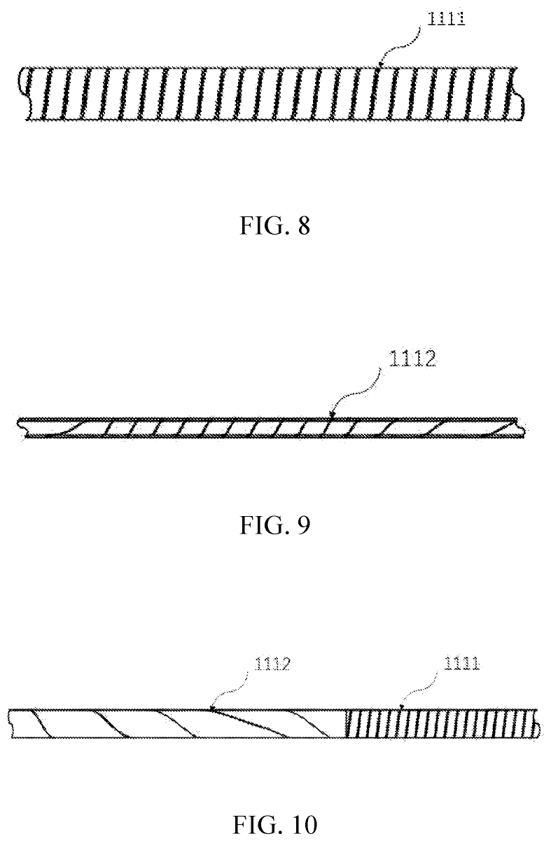

In combination with the embodiments referred to in FIGS. 1-7, and referring to FIG. 8, in some embodiments, the spring tube 111 consists of a metal wire wound spring tube 1111.

In combination with the embodiments referred to in FIGS. 1-7, and referring to FIG. 9, in some embodiments, the spring tube 111 consists of a spirally cut stainless steel tube 1112.

In combination with the embodiments referred to in FIGS. 1-7, and referring to FIG. 10, In some typical embodiments, the spring tube 111 consists of a metal wire wound spring tube 1111 and a spirally cut stainless steel tube 1112, wherein the spirally cut stainless steel tube 1112 is a hypotube, and one end of the metal wire wound spring tube 1111 is connected to the spirally cut stainless steel tube 1112, both are welded as a whole at the end face where they contact. According to a cold snare 100 provided in the present embodiment, a working principle of the cold snare 100 is as follows:

during use, the distal end of the cold snare 100 is inserted into the human body through the endoscopic forceps channel, and the handle 120 is used to control the drive wire 130 to manipulate the loop 140. In some typical embodiments, by pushing and pulling the front handle 121 to slide relative to the rear handle 122, the drive wire 130 is driven to slide relative to the spring tube 111 in the direction of the longitudinal axis 170, thereby manipulating the loop 140 to enter and exit the spring tube 111 so that the loop 140 is either in an open state (as shown in FIG. 2 or 5) or a retracted state (as shown in FIG. 6). When the loop 140 is in the open state, the user can perform the operation of enclosing polyps, and when the loop 140 switches from the open state to the retracted state, the radial dimension of the loop 140 is reduced to cut the polyps. At the same time, during the enclosing process, the drive wire 130 can be driven to rotate relative to the spring tube 111 by rotating the handle 120, and then the loop 140 is rotated relative to the spring tube 111, so as to adjust the angle of the loop 140, and the loop 140 has high rotational synchronization.

A cold snare 100 provided by the present embodiment has at least the following advantages:
the cold snare 100 provided in the embodiments of the present disclosure uses the spring tube 111 as the outer tube and take advantage of high strength characteristic of the spring tube 111, to enable complete removal when cold cutting a larger polyp. At the same time, use of nickel-titanium monofilament in the loop 140 makes the loop 140 maintain the water drop shape while changing the radial dimension, to enclose polyps at any time at different radial dimensions and solve the problem of using snares with different sizes for different sizes of polyps in the same operation process, helping to shorten the operation time and reduce the operation cost; moreover, the nickel-titanium monofilament has larger strength than the multi-stranded stainless steel wires, and thinner diameter than the multi-stranded stainless steel wires, which can better realize the cutting of polyps, and facilitates cutting of larger size polyps, thereby enabling the cold snare 100 with nickel-titanium monofilament provided in the present embodiment to cut large size polyps (such as 25 mm).

The above is only the embodiments of the present disclosure, but the scope of protection of the present disclosure is not limited to this, any change or replacement readily conceivable by technical person familiar with this technical field shall be covered within the scope of protection of the present disclosure. Therefore, the scope of protection of the present disclosure shall be subject to the scope of protection of the claims.

### INDUSTRIAL PRACTICABILITY

The cold snare provided by the present disclosure can realize the reciprocating motion of the drive wire in the direction of longitudinal axis of the spring tube, and the synchronous reciprocating motion of the loop with the drive wire in the direction of the longitudinal axis of the spring tube, and thus achieve the purpose of manipulating the loop to enter and exit the spring tube by the drive wire. In the present disclosure, the spring tube is used as the outer tube, and the strength of the spring tube is higher than that of the plastic outer tube of the existing cold snare, so it is helpful for the loop to cut when grabbing large polyps. At the same time, since the spring tube is made of metal, the friction generated by the spring tube when the loop is rotated is less than the friction when the existing plastic outer tube contacts the loop, so it is helpful to realize the synchronous and precise rotation of the loop, which is convenient for the doctor to accurately enclose the polyp in an operation, improve the use effect, having excellent practical performance.

What is claimed is:

1. A cold snare, comprising:
a spring tube (111);
a drive wire (130), passing through the spring tube (111), and performing a reciprocating motion in a direction of a longitudinal axis (170) of the spring tube (111); and
a loop (140), fixedly connected to a distal end of the drive wire (130), and motion of the drive wire (130) being used to manipulate the loop (140) to enter and exit the spring tube (111).

2. The cold snare according to claim 1, wherein the cold snare (100) further comprises an end member (150) fixedly connected to a distal end of the spring tube (111), the end member (150) has an inner hole (151) in communication with the spring tube (111); the loop (140) enters and exits the spring tube (111) through the inner hole (151).

3. The cold snare according to claim 2, wherein a proximal end of the end member (150) holds the distal end of the spring tube (111) and a distal end of the end member (150) is a contraction port.

4. The cold snare according to claim 2, wherein a distance between a merger point (148) and an opening (149) of the loop (140) is 5-15 mm.

5. The cold snare according to claim 2, wherein the end member (150) has a distal end face (153) and an inner circumferential surface corresponding to the inner hole (151), and a round corner (152) is arranged between the distal end face (153) and the inner circumferential surface.

6. The cold snare according to claim 2, wherein the end member (150) is made of a metal.

7. The cold snare according to claim 1, wherein the loop (140) is made of a nickel-titanium alloy.

8. The cold snare according to claim 7, wherein the loop (140) is a single wire structure.

9. The cold snare according to claim 8, wherein the wire has a round, square or flat cross section.

10. The cold snare according to claim 7, wherein the loop (140) comprises a first ring segment (143), a necking segment (144) and a second ring segment (145) connected sequentially; the first ring segment (143) has a first intersection point (146) connected with the necking segment (144), and the first ring segment (143) is in a streamlined shape with smooth transition from a proximal end of the first ring segment (143) to the first intersection point (146); the second ring segment (145) has a second intersection point (147) connected with the necking segment (144), and the second ring segment (145) is in a streamlined shape with smooth transition from a proximal end of the second ring segment (145) to the second intersection point (147).

11. The cold snare according to claim 7, wherein the loop (140) is in water drop or oval shape.

12. The cold snare according to claim 1, wherein the cold snare (100) further comprises a riveted tube (160), an outer circumferential surface of the riveted tube (160) is a cylindrical surface, the distal end of the drive wire (130) is fixedly connected into the riveted tube (160), and a proximal end of the loop (140) is fixedly connected into the riveted tube (160).

13. The cold snare according to claim 1, wherein the cold snare (100) further comprises an outer skin (112) cladding on outside of the spring tube (111).

14. The cold snare according to claim 13, wherein the outer skin (112) comprises a polymer tube.

15. The cold snare according to claim 1, wherein a distal end of the spring tube (111) is further provided with a scale (113) for measuring a size of a polyp to be cut.

16. The cold snare according to claim 1, wherein the spring tube (111) comprises a flexible metal tube or an elastic metal tube.

17. The cold snare according to claim 16, wherein the flexible metal tube or the elastic metal tube comprises any one of a metal wire wound spring tube (1111) and a spirally cut metal tube such as a spirally cut stainless steel tube (1112), or a combination of them.
